(19) United States
(12) Patent Application Publication
(10) Pub. No.: US 2022/0017833 A1
(43) Pub. Date: Jan. 20, 2022

(54) Methods and Compositions for Scavenging Sulfides from Hydrocarbon Fluids and Aqueous Streams

(71) Applicants: James Begeal, Cibolo, TX (US); Chris Williamson, Montgomery, TX (US); Ali Yousef, Tomball, TX (US)

(72) Inventors: James Begeal, Cibolo, TX (US); Chris Williamson, Montgomery, TX (US); Ali Yousef, Tomball, TX (US)

(73) Assignee: Nexgen Oilfield Chemicals, LLC, Cypress, TX (US)

(21) Appl. No.: 17/072,594

(22) Filed: Oct. 16, 2020

Related U.S. Application Data
(60) Provisional application No. 62/916,714, filed on Oct. 17, 2019.

(51) Int. Cl. C10L 3/10; C10G 21/16; C10G 29/22; C02F 1/20

(52) U.S. Cl. CPC C10L 3/103; C10G 21/16; C10G 29/22; C02F 2101/101; C10L 2290/545; C10G 2300/207; C02F 1/20

(57) ABSTRACT

Embodiments of a composition of the present invention for scavenging sulfides from hydrocarbon fluids and water generally include diaminol/diaminacetal provided in a chemical system, wherein the diaminol/diaminacetal is prepared by reacting one molar equivalent of glyoxal and two molar equivalents of a primary amine functionality. In various embodiments, the chemical system includes at least one component selected from surfactants, hydrotropes, alcohols, amines, amino acids, and ethers. Embodiments of a method for scavenging sulfides from hydrocarbon fluids and water is also provided.

### CROSS-REFERENCE TO RELATED APPLICATIONS

This application claims the benefit of U.S. Provisional Application No. 62/916,714, filed on Oct. 17, 2019, which application is incorporated herein by reference as if reproduced in full below.

### STATEMENT REGARDING FEDERALLY SPONSORED RESEARCH OR DEVELOPMENT

Not Applicable.

### FIELD OF THE INVENTION

The present invention generally relates to removal of impurities from hydrocarbon fluids and aqueous streams. More particularly, embodiments of the present invention are directed to compositions and methods for scavenging sulfides from oil, natural gas and water. In one embodiment, such compositions comprise an imine and/or a diimine provided in a chemical system, wherein the imine and/or diimine is converted into the form of an aminol and/or diaminol and/or its corresponding aminacetal and/or diaminacetal.

### BACKGROUND OF THE INVENTION

Oil and natural gas occur naturally in geologic formations beneath the earth's surface and often contain water, carbon dioxide (CO₂), and sulfur (including hydrogen sulfides and organic sulfides). Oil, natural gas or water containing sulfur is considered to be “sour.” Hydrogen sulfide and organic sulfides (hereinafter “sulfides”) are malodorous and toxic. The concentration of hydrogen sulfide (H₂S) in natural gas may range from 0.1 ppm to greater than 150,000 ppm.

Hydrogen sulfide, which is toxic, is corrosive in gaseous form, and may corrode steel piping during transport and storage. Because of the corrosivity and toxicity of sulfides, producers of natural gas and oil typically remove the sulfides from the extracted hydrocarbon and water streams separated therefrom. In some cases, sulfides are removed from the hydrocarbon streams in the wellbore, at (or near) the wellhead, and/or within inter-field storage of crude products or transfer equipment therefor (i.e., “upstream”). In other cases, sulfides are removed from the hydrocarbon fluids and water streams after transport of the crude stream from the upstream sector to the “midstream” sector, where fluid separation may take place. In other cases, sulfide impurities are removed from the hydrocarbon fluids and/or water streams during the refining process, i.e., “downstream.” In one aspect, impurity removal may be performed at more than one of these process locations. Hydrocarbons or water streams from which sulfides have been removed are considered to be “sweet.”

Upstream and midstream processing plants, employed to strip the sulfides from the oil and natural gas stream, or water streams, before refining, may be located in producing oil and natural gas fields. Upstream processing of hydrocarbons typically comprises treating a mixture of oil and natural gas, but may comprise treating the separated natural gas. Midstream processing typically comprises individually treating the oil and/or natural gas which have been substantially separated. In one aspect, oil and/or natural gas processing plants may utilize bubble columns, packed columns, tray columns, and/or other methods to absorb sulfides into a liquid. The spent liquid, comprising fully or mostly reacted scavenger, is then typically blown down to a wastewater facility and replenished with fresh scavenger. Fouling in the treated line, contact tower, or wastewater stream caused by insoluble solid reaction products is one of the limitations of using chemical sulfide scavengers. In another aspect, the oil and/or natural gas may be exposed to sulfide “scavengers” by being sprayed with the liquid scavenger through an atomizing injection nozzle. The scavenger destructively reacts with the sulfides or otherwise removes the sulfides from the hydrocarbon fluid. The sprayed and spent scavenger must then be carried downstream by the treated stream without fouling any downstream equipment vessel or process. Chemical scavengers, which historically include triazines, formaldehyde, and glyoxal compositions, chemically remove these impurities from the hydrocarbon fluid by irreversible reactions. The reaction products (spent scavenger) must then be dealt with in some manner. Examples of such chemical hydrogen sulfide scavenging are disclosed in, for example, U.S. Pat. No. 5,169,411 to Weers; U.S. Pat. No. 7,985,881 to Westlund et al.; U.S. Pat. No. 10,093,868 to Weers; U.S. Pat. No. 10,196,343 to Harrington et al.; U.S. Pat. No. 10,308,886 to Rana et al.; U.S. Pat. No. 10,119,079 to Fuji et al.; U.S. Pat. No. 10,294,428 to Suzuki et al.; and U.S. Pat. No. 10,513,662 to Weers et al., U.S. Patent Application Publication No. 2020/0024526 by Weers et al., and U.S. Patent Application Publication No. 2019/0322948 by Begeal et al., each of which is incorporated herein by reference in their entirety to the extent not inconsistence herewith.

A traditionally preferred class of scavenger comprises triazines, particularly those formed by the reaction of formaldehyde and monoethanolamine. Triazines are traditionally viewed as safer than formaldehyde, having a good scavenging capacity, and having a lower cost compared to other scavengers. Importantly however, triazines are made with and can contain free formaldehyde—a probable human carcinogen—which poses health concerns. More problematically, upon reaction with sulfides, triazines typically form solids that form deposits in pipes, equipment, and wastewater stream, which adds to maintenance costs and downtime expenses of lost production. Even with these undesirable side effects with the employment of triazines as sulfide scavengers, oil and natural gas producers have generally found it necessary to utilize triazines, as there has not been another suitable option available for upstream or midstream-production, especially in the aqueous and gas phase of the production stream.

Another class of compounds used as a sulfide scavenger comprises diimines. Examples of this technology are described in previously mentioned U.S. Pat. No. 5,169,411 to Weers and U.S. Pat. No. 9,394,396 to Stark et al.; which are incorporated herein by reference in their entirety to the extent not inconsistence herewith. Unfortunately, the diimine technology to date for sulfide scavenging suffers from the same problem of solids formation as described above regarding triazine technology. Additionally, the diimines currently utilized display very poor scavenging reaction kinetics; that is, in the relatively short timeframe available for sulfide scavenging these diimines are not able to efficiently scavenge the sulfides and levels thereof in the hydrocarbon fluids cannot be sufficiently reduced. Moreover, traditional diimine scavenger production has required that once the reaction of an aqueous glyoxal solution with a monoamine is carried out, it is necessary to separate the water from the water-insoluble diimine to provide a useful active agent composition. Reaction of aqueous glyoxal with a molar excess of polyamine leaves free amine groups on the polyimine to impart initial water solubility, but the spent polyimine (the reaction product with hydrogen sulfide) forms polymeric solids much more readily. Similarly, reaction of monoamines with hydroxy-aldehydes leaves free hydroxyl groups on the imine to impart water solubility, but these non-reactive groups reduce its scavenging capacity. Reacting mono-aromatic aldehydes with alkanolamines also leaves free hydroxyl groups on a more polar mono-imine to impart some water solubility, but this also reduces scavenging capacity and still produces insoluble solids when reacted with hydrogen sulfide. Accordingly, a suitable sulfide scavenger that has suitable solubility properties, works without forming solids, and exhibits satisfactory reaction kinetics is desirable.

### BRIEF SUMMARY OF THE INVENTION

The present invention is directed to compositions and methods for removing sulfides (hydrogen sulfide and organic sulfides) from oil, natural gas, and water, without the formation of insoluble solids, using aminols and/or aminacetals in mixed alcohol solvents. Embodiments include precursor compositions comprising diimines, such as, but not limited to, a diimine formed by the reaction of one molecule of a di-aldehyde, such as, but not limited to, glyoxal, and two molecules of a primary amine, wherein such precursors are then reacted with water and/or alcohols to convert that adduct into a more reactive, water-soluble aminol or aminacetal, which may be used to scavenge sulfides from aqueous and hydrocarbon fluids. Additional embodiments of sulfide scavengers comprise precursor imines, such as, but not limited to, an imine formed by the reaction one molecule of a mono-aldehyde with one molecule of a primary amine, followed by reaction with water and/or alcohols to convert the precursor imine into a more reactive water-soluble aminol or aminacetal which may be used to scavenge sulfides from aqueous and hydrocarbon fluids. Additional embodiments of sulfide scavengers comprise specific mixed alcohol systems wherein each aldehyde group of a mono- or di-aldehyde is reacted with one molecule of a primary or secondary amine to produce water-soluble aminols and/or aminacetals which may be used to scavenge sulfides from aqueous and hydrocarbon fluids. One embodiment comprises reaction products of glyoxal, isopropylamine, water, water-miscible alcohols, and water-immiscible alcohols. Embodiments of a method of scavenging sulfides with the compositions are also provided.

### BRIEF DESCRIPTION OF THE DRAWINGS

For a more complete understanding of the present invention, reference is now made to the accompanying drawings, in which:

FIG. 1 is a schematic depiction of a chemical testing system used to evaluate embodiments of a sulfide scavenger of the present invention.

FIG. 2 is a schematic depiction of a chemical spray testing system used to evaluate embodiments of a sulfide scavenger of the present invention.

FIG. 3 graphically depicts molar capacity against molar concentration.

### DETAILED DESCRIPTION OF EXEMPLARY EMBODIMENTS OF THE INVENTION

The exemplary embodiments are best understood by referring to the drawing and the written description. The present disclosure is directed to compositions and methods of using one or more aminols and/or diaminols, or the corresponding aminacetals and/or diaminacetals, as a component of a sulfide scavenger system. As used herein, the term H₂S may be used interchangeably with the term sulfide(s), unless the context indicates otherwise. As used herein, the term “aminol” refers to a molecule in which an amino group and a hydroxyl group are attached to the same carbon. As used herein, the term “aminacetal” refers to a molecule in which an amino group and an alkoxy group are attached to the same carbon. Such molecules, having the general structure depicted in Structure 1, wherein for an aminol R³ is hydrogen, and for an aminacetal R³ is an alkyl (or other moiety wherein a carbon atom is linked to the oxygen atom), as shown below:

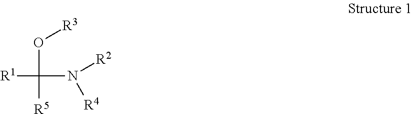

In various embodiments, each of the R², R⁴ and R⁵ moieties depicted in Structure 1 may be hydrogen, alkyl, or any other useful substituent, as would be understood by one skilled in the art.

In one embodiment, an aminol of the present invention may be provided by reacting a molecule comprising at least one aldehyde functional group with a molar equivalent of a primary or secondary amine functionality in a water miscible alcohol as depicted in Equation 1 below.

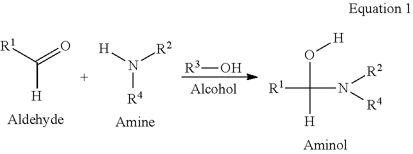

The aminacetal of the present invention is then formed in equilibrium with the corresponding aminol, as depicted in Equation 2 below.

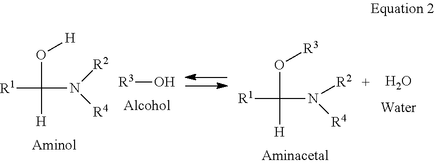

In one embodiment, an aminol and/or aminacetal of the present invention may be provided by reacting a molecule comprising at least one aldehyde functional group with a molar equivalent of a primary amine functionality to produce a two-phase mixture of an imine and water, as depicted in Equation 3 below. In one aspect, the so-produced imine may be reacted with water to form an aminol. In one embodiment, such a reaction may be carried out in a solvent comprising an alcohol or mixture of alcohols co-miscible with the water and the imine to produce a single-phase solution of the aminol in equilibrium with an aminacetal, in an aqueous alcoholic solvent, as depicted in Equation 4 below. In another aspect, the so-produced imine may react with alcohol to form an aminacetal. In one embodiment, such a reaction may be carried by first removing the water, then adding an alcohol or mixture of alcohols miscible with the imine, to produce a single-phase solution of the aminacetal, as depicted in Equation 5 below. In another embodiment, the aldehyde, a primary or secondary amine, and an alcohol are reacted to produce a single-phase solution of the aminol in equilibrium with an aminacetal in an aqueous alcohol solvent, as depicted in Equation 6 below.

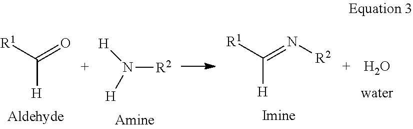

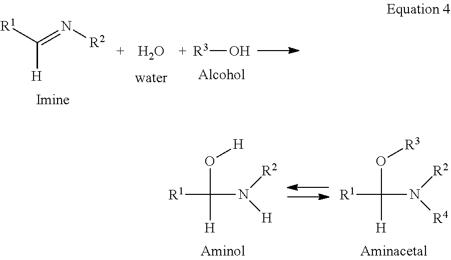

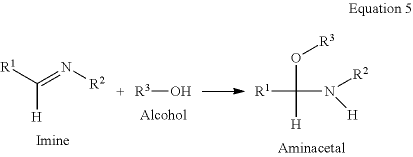

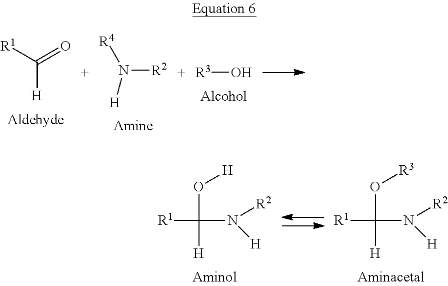

In one aspect, aldehydes useful in providing precursor imines employable in embodiments of the present invention include, but are not limited to, saturated mono-aldehydes, such as formaldehyde, acetaldehyde, propionaldehyde, valeraldehyde, and butyraldehyde; conjugated mono-aldehydes, such as benzaldehyde, 2,3 dimethylbenzaldehyde, 2-ethoxybenzaldehyde, cinnamaldehyde, and acrolein; saturated di-aldehydes, such as malondialdehyde, succinaldehyde, and glutaraldehyde; and conjugated di-aldehydes, such as phthalaldehyde, malealdehyde, fumaraldehyde, and glyoxal. In various embodiments, primary and secondary amines such as, but not limited to, the amines listed in Table 1 below may be employed to produce imines, aminols, and/or aminacetals suitable for use in embodiments of the present invention.

As used herein the terms “diimine” (also referred to in the art as “di-imine” or “bis-imine”) “diaminol” (also referred to in the art as “di-aminol” or “bis-aminol”) and “diaminacetal” (also referred to in the art as, “di-aminacetal” or “bis-aminacetal”), mean a molecule comprising at least two separate carbon-nitrogen bonds, and having the general structures depicted in Structures 2, 3, and 4 below, respectively:

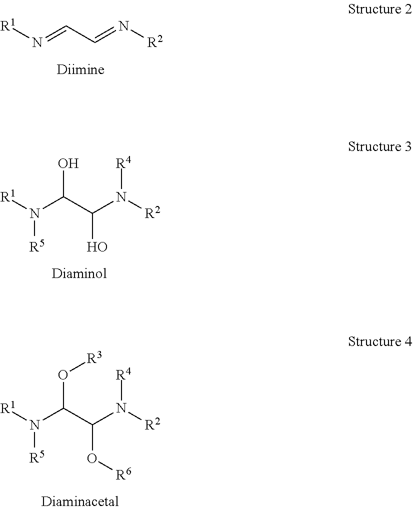

In various embodiments, the diimines, diaminols, and diaminacetals of the present invention contain only the two adjacent carbon atoms (connected by the single bond specifically depicted in Structures 2, 3 and 4) between the nitrogen atoms, and each of these carbon atoms has a hydrogen substituent, although the invention is not so limited and three or more carbon atoms and/or carbon atoms containing substituents that are not hydrogen may be situated between the nitrogen atoms. Exemplarily, unsaturated connecting carbons in conjugation with the two aldehyde groups, such malealdehyde and fumaraldehyde, are included. In various embodiments, each of the R¹, R², R⁴, R⁵, R⁶ moieties depicted in Structures 2, 3, and 4 may be alkyl or any other useful substituent, as would be understood by one skilled in the art. In various embodiments, R¹, R², R⁴, and/or R⁵ may also be hydrogen.

In one embodiment, a diaminol or diaminacetal of the present invention may be provided by reacting a molecule comprising two aldehyde functional groups (a “dialdehyde”) with two molar equivalents of a primary amine functionality to produce a two-phase mixture of a diimine and water. In one aspect, the so produced diimine may be reacted with water to form a diaminol. In one embodiment, such a reaction may be carried out in a solvent comprising an alcohol or mixture of alcohols co-miscible with the water and the diimine, to produce a single-phase solution of the diaminol in equilibrium with the diaminacetal in an aqueous alcoholic solvent. In another aspect, the so produced diimine may react directly with an alcohol to form a diaminacetal. In one embodiment, such a reaction may be carried by first removing the water, then adding an alcohol or mixture of alcohols miscible with the diimine, to produce a single-phase solution of the diaminacetal. In another embodiment, two molar equivalents of a primary or secondary amine functionality may be reacted with the dialdehyde and an alcohol to directly produce a single-phase solution of the diaminol in equilibrium with the diaminacetal in aqueous alcoholic solvent. In one embodiment, the two molar amine equivalents may comprise two molecules of a compound comprising a single amine or amine functionality, although the invention is not so limited and mixed amines and multiple amine functionality source compound(s) may be employed.

Glyoxal, and its diimines, diaminols, and diaminacetals, have both cis (same side) and trans (opposite side) conformations of the carbon substituents relative to the carbon-carbon bond axis. The interconversion of cis and trans glyoxal in aqueous solution via the hydrate is shown in Equation 7.

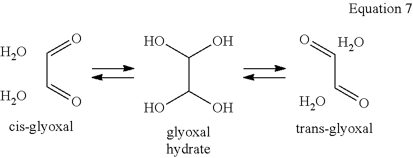

An example of the cis conformation of glyoxal's isopropyl diaminol is shown in Structure 5, with the trans conformation shown in Structure 6 (both below). When glyoxal is employed as the dialdehyde in the present invention, the close proximity of the adjacent carbons can interfere with simultaneous attack on both carbons when the leaving groups are on the same side of the carbon-carbon bond axis, as they are in the cis conformation. The axis of nucleophilic attack by sulfide toward the C—N bond in cis- and trans-glyoxy diisopropyl aminol is shown by arrows in Structures 5 and 6, respectively. As one skilled in the art would understand, the trans conformation (Structure 6) allows simultaneous attack at both sites from different places, whereas the cis conformation (Structure 5) has only a single spot from which to attack either site, but not both sites simultaneously. Not to be bound by theory, this is thought to allow the trans conformation to scavenge sulfide more quickly.

The cis conformation also has the ability and tendency to undergo intramolecular condensations over time, of the type known to occur in its glyoxal precursor. One example is the cis-glyoxy-diisopropyl aminol dimer condensate shown in Structure 7. This secondary cis condensation blocks the reactive sites, degrading the scavenging capacity over time.

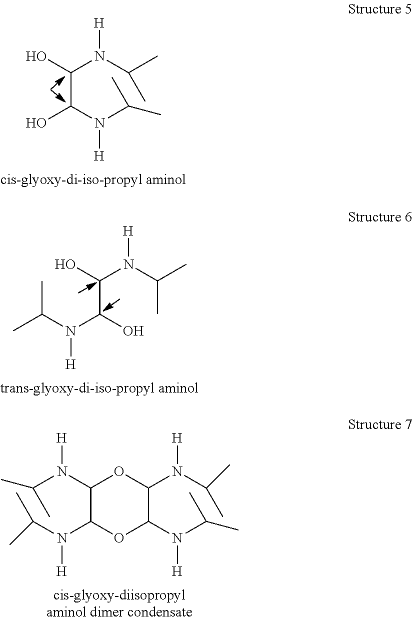

In some embodiments, the carbon-carbon bond axis from the glyoxal is free to rotate between the cis and trans conformations, so there exists an equilibrium distribution therebetween. In some embodiments, glyoxal is added to amines in which at least one of the alkyl groups on both amines has alpha-branching. In such an embodiment, the crowding or steric hindrance caused by alpha branching on both amines prevents free rotation to the cis conformation, causing the molecule to exist predominantly in the trans conformation. The crowding of the twin isopropyl groups hindering the formation of cis-glyoxy-diisopropyl aminol is shown in Structure 5, contrasted with the lack of crowding in the trans-glyoxy-diisopropyl aminol (as shown in Structure 6) which favors formation thereof.

The steric hindrance imparted by each of these exemplary amines in terms of the presence or absence of branching on the first or “alpha” carbon attached to the nitrogen is noted in Table 1. “Alpha-branching” refers, in Structure 8 below, to at most one of R², R³, and R⁴ being hydrogen, and therefore at least two thereof being an alkyl or other substituent. With a primary amine, R¹ in Structure 8 represents a hydrogen atom. With a secondary amine, R¹ represents an alkyl or other substituent. In one aspect, for an embodiment utilizing a secondary amine, only one of the alkyls on each amine would need to be alpha branched to meet the steric hindrance requirement. In various embodiments, it is not necessary that the amine be fully “hindered” in the usual sense of blocking reactive access to the adjacent carbon, as, for example, the tert-butyl group is often used to accomplish, only that the alkyl has a single alpha branch, enough to interfere somewhat with the formation of the cis configuration of the adjacent carbons.

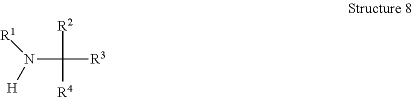

In one embodiment, depicted in Equation 8 below, a diimine precursor of the present invention is provided by reacting one molar equivalent of glyoxal [oxaldehyde] with two molar equivalents of isopropyl amine [propan-2-amine].

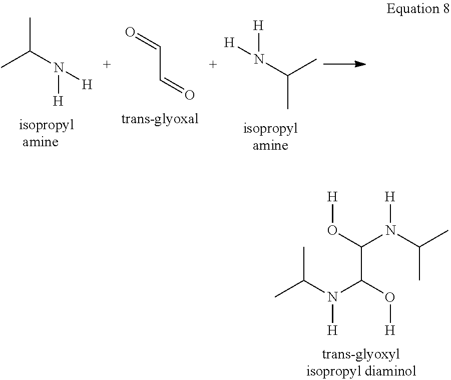

The reaction product, 1,2-bis[(propan-2-yl)amino]ethane-1,2-diol, hereinafter referred to as “glyoxy isopropyl diaminol” (GIDA), is merely exemplary, as described further herein. Dehydrating this reaction product (removing water, Equation 3) produces the imine [N,N′-di(propan-2-yl)ethane-1,2-diimine, CAS No. 24764-90-7], hereinafter referred to as “glyoxal isopropyl diimine” (GID). Replacing the water with an alcohol (Equation 5) produces the corresponding aminacetal, hereinafter referred to as “glyoxy isopropyl diaminacetal,” (GIDAc). In various embodiments, other dialdehydes, such as, but not limited to malondialdehyde [OCHCH2CHO], succindialdehyde [OCHCH₂CH2CHO], glutaraldehyde [OCHCH₂CH₂CH₂CHO], malealdehyde [cis-OCHCH═CHCHO], fumaraldehyde [trans-OCHCH═CHCHO], and substituted forms thereof may be employed. In addition, in other embodiments, other primary and secondary amines (or mixtures thereof), such as, but not limited to, the primary and secondary amine compounds listed in Table 1 may be employed.

TABLE 1

| Structural Formula | Traditional Name | Preferred IUPAC Name | α-Branched |
|---|---|---|---|
| CH₃NH₂ | methylamine | methanamine | No |
| CH₃CH₂NH₂ | ethylamine | ethanamine | No |
| CH₃(CH₂)₂NH₂ | normal-propylamine | propan-1-amine | No |
| (CH₃)₂CHNH₂ | iso-propylamine | propan-2-amine | Yes |
| (CH₂)₂CHNH₂ | cyclo-propylamine | cyclopropanamine | Yes |
| CH₃(CH₂)₃NH₂ | normal-butylamine | butan-1-amine | No |
| (CH₃CH₂)CH₃CHNH₂ | sec-butylamine | butan-2-amine | Yes |
| (CH₃)₂CHCH₂NH₂ | iso-butylamine | 2-methylpropan-1-amine | No |
| (CH₃)₃CNH₂ | tert-butylamine | 2-methylpropan-2-amine | Yes |
| (CH₂)₅CHNH₂ | cyclohexylamine | cyclohexanamine | Yes |
| HOCH₃CH₂NH₂ | ethanolamine | 2-aminoethanol | No |
| CH₃CHOHNH₂ | 1-aminoethanol | 1-aminoethanol | Yes |
| OH(CH₃)CHCH₂NH₂ | iso-propanolamine | 1-aminopropan-2-ol | No |
| HO(CH₂)₃NH₂ | normal-propanolamine | 3-amino-1-propanol | No |
| (CH₃)₂NH | dimethylamine | n-methylmethanamine | No |
| CH₃(CH₃CH₂)NH | methylethylamine | n-methylethanamine | No |
| (CH₃CH₂)₂NH | diethylamine | n-ethylethanamine | No |
| (CH₃)(CH3)2CHNH | methylisopropylamine | n-methyl-2-propanamine, | Yes |
| ((CH₃)₂CH)₂NH | diisopropylamine | n-2-propyl-2-propanamine, | Yes |
| (HOCH₃CH₂)₂NH | diethanolamine | 2,2′-aminodiethanol | No |
| CH₃(HOCH₃CH₂)NH | methylethanolamine | 2-(methylamino)ethanol | No |
| (OH(CH₃)CHCH₂)₂NH | diisopropanolamine | 1-(2-hydroxypropylamino)propan-2-ol | No |

Additional examples of amines suitable for use as described herein include, but are not limited to, aromatic amines, such as aniline, and methyl anthranilate; and ammonia. Diamines and higher multiple-amine compounds, which have two or more amine groups in the molecule, may also be used. Non-limiting examples include ethylenediamine, diethylenetriamine, triethylenetetramine, and tetraethylenepentamine.

Generally, imine and diimine precursors may be produced as is well known in the art. In one aspect, diimine precursors may be produced as disclosed in U.S. Pat. No. 7,985,881 to Westlund et al., which is incorporated herein by reference in its entirety to the extent not inconsistence herewith. In one embodiment of the present invention, glyoxal isopropyl diimine [N,N′-di(propan-2-yl)ethane-1,2-diimine] (GID), a precursor to the diaminol and/or diaminacetal, is produced by providing one molar equivalent of glyoxal (as a 40% aqueous solution thereof) in a reactor vessel and controllably introducing two molar equivalents of isopropyl amine, neat, such that the pressure in the reaction vessel is maintained below about 20 PSI and the temperature in the reaction vessel is maintained below about 110° F., until a two-phase reaction product forms. In one embodiment, the two-phase reaction product of diimine and water is then co-solubilized by adding a water immiscible alcohol such as, but not limited to C₄ to C₁₂ alcohols, in an amount equal to from 15 to 30% of the water-insoluble imine phase, which adds to the imine phase, whereupon, a water miscible alcohol, such as, but not limited to, methanol, is added until a one-phase mixture of glyoxyl diaminol/diaminacetal (GIDA/GIDAc) is created. In one embodiment, the amount of alcohol added is equal to from about 20% to 60% of the final solution volume. In one embodiment, the formula percent weighted ratio of carbon to oxygen (C/O) in the mixed alcohol may be in the range of 1.0 to 2.0. For example, if the mixed alcohol consists of octanol (C/O=8) at 10% of the total solution, and methanol (C/O=1) at 23% of the total solution (the remainder being the imine and water), the formula percent weighted total C/O in the mixed alcohol 10%×8.0+23%×1.0=1.03. In other embodiments, the reaction which produces the one-phase mixture of glyoxyl isopropyl diaminol/diaminacetal (GIDA/GIDAc) is carried out directly in a solvent comprising a water miscible alcohol such as, but not limited to, methanol, and a water immiscible alcohol, such as, but not limited to C₄ to C₁₂ alcohols. In one embodiment, the mixed alcohol solvent used for this direct one-step reaction has the same characteristics as the two-step process above. Although the reaction alcohol solvent mixture is exemplarily described above for the one-step or two-step process, the invention is not so limited and other alcohol mixture ratios may be employed. In one aspect, a resulting diaminol/diaminacetal reaction product (the “active component”) may be maintained in the aqueous alcoholic solvent system and used as is. In another aspect, an initial water insoluble imine reaction product may be separated from the water (such as by decanting), and then the solvent comprising an essentially anhydrous but water miscible alcohol such as, but not limited to, methanol, and an essentially anhydrous but water immiscible alcohol, such as, but not limited to C₄ to C₁₂ alcohols, is added in an amount that the overall composition remains water miscible. The resultant imine/aminacetal (the “active component”) may be maintained in the essentially anhydrous, overall water miscible mixed alcoholic solvent system and used as is.

In various embodiments, suitable water-miscible alcohols comprise those with a carbon to oxygen ratio (C/O) of no more than 3.0 and an octanol/water partition coefficient K_{ow} (“P”), as the logarithm (LogP) of from about −1.4 to 0.8, although the invention is not so limited. Such alcohols found useful in embodiments of the present invention include, without limitation, methanol, ethanol, isopropanol, butoxy ethanol, propylene glycol, and hexylene glycol.

In various embodiments, useful water-immiscible alcohols include those with a carbon to oxygen ratio (C/O) of no less than 4.0 and an octanol/water partition coefficient P, as the logarithm (LogP), of from 0.9 to 5.0, although the invention is not so limited. In various embodiments, such alcohols found useful in the present invention include, without limitation, n-butanol, n-heptanol, n-octanol, 2-ethylhexanol, and Neodol® 91 (mixture of C₉-C₁₁ linear alcohols).

In various embodiments, additional water-miscible alcohols or common salts, such as, but not limited to, sodium chloride or ammonium sulfate, can also be added, in a manner known in the art, to suppress the freezing point or pour point to achieve usable cold flow properties for different field applications.

In one embodiment, diaminols and/or diaminacetals of the present invention may be provided as a component of a chemical system together with components such as, but not limited to, surfactants, dispersants, hydrotropes, additional alcohols and amines, demulsifiers, corrosion and scale inhibitors, and/or other sulfide scavengers in a natural gas, oil, or water stream in upstream or midstream production environments. The diaminol and/or diaminacetal blend may be formed in-situ, such as in process equipment or in a wellbore or underground formation, or formed ex-situ and injected or pumped into the natural gas, oil, or water stream.

In one aspect, a chemical system in which embodiments of the diaminols/diaminacetals of the present invention may be useful may comprise surfactants such as, but limited to, non-ionic surfactants, including, but not limited to, alcohol ethoxylates, alkylphenol alkoxylates, alkylphenol-formaldehyde resin alkoxylates, polyol alkyloxylates, polyglucosides, amine oxides, polyol fatty esters and amides; anionic surfactants, including carboxylates, sulfonates, sulfates, phosphonates and phosphates, and alkoxylates thereof; cationic surfactants, including fatty amines and ammoniums, imidazolines and imidazoliniums, phosphoniums, and alkoxylates thereof.

In one aspect, chemical systems in which embodiments of the diaminols/diaminacetals of the present invention may be useful may comprise dispersants such as, but limited to, nonionic dispersants, including, but not limited to, polysaccharides, polyvinylalcohols, polyhydroxystearic esters, and polyisobutylene succinic esters; anionic dispersants, including polyacrylates, polyvinylcarboxylates, polysaccharidic carboxylates, polyvinylsulfonates, polyacrylosulfonates, polystyrenesulfonates, polynaphthalene-formaldehye sulfonates, polyaminophosphonates, and polyvinylphosphonates; and cationic dispersants, including, but not limited to, polyamines and polyammoniums, polyvinylamines, polyvinylammoniums, polyacryloamines and polyacryloammoniums, and polyaminosaccharides.

In one aspect, a chemical system in which embodiments of the diaminols/diaminacetals of the present invention may be useful may comprise hydrotropes such as, but not limited to, sodium, potassium or ammonium salts of sulfonates of benzene and alkyl benzenes, cumene, naphthalene and alkyl naphthalenes, and unsaturated oils; fatty dimer/trimer carboxylic acids and their salts, and phosphate partial esters; glymes and other polyglycol diethers.

In one aspect, a chemical system in which embodiments of the diaminols/diaminacetals of the present invention may be useful may comprise alcohols beyond those in the primary mix alcohol reaction solvent, such as, but not limited to, primary and secondary alcohols; ethylene, diethylene, and polyethylene glycols; propylene, dipropylene, and polypropylene glycols, glycerol, sorbitol, pentaerythritol, and mono-ethers of glycols and polyglycols.

In one aspect, a chemical system in which embodiments of the diaminols/diaminacetals of the present invention may be useful may comprise additional amines beyond those used to make the diaminols/diaminacetals such as, but not limited to, monoethanolamine (MEA), diethanolamine (DEA), methyl diethanolamine (MDEA), diisopropanolamine (DIPA), and/or aminoethoxyethanol/diglycolamine (DGA).

### Testing

Prospective scavengers for gas applications were evaluated using eight separate tests:
Long Reaction Time Capacity
Short Reaction Time Capacity
Storage Capacity Loss
Solution Homogeneity
Hot Spray Fouling
Hot Spray Degradation
Spent Solids Fouling
Spent Water Flowability

### Test Apparatus

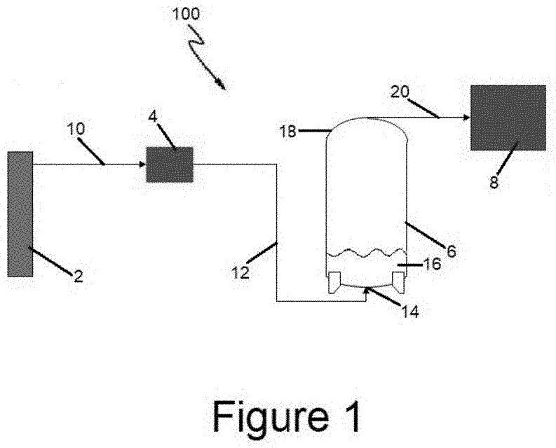

Referring now to FIG. 1, therein is schematically depicted a sulfide scavenger testing system 100 comprising a gas source 2, a flow regulator 4, gas treatment vessel 6, and a sulfide measurement device 8. In one testing embodiment, gas (not shown) is flowed from gas source 2 via gas feed piping 10 into flow regulator 4, which regulates gas flow to gas treatment vessel 6 via regulated gas feed piping 12. The regulated gas flows into gas treatment vessel 6 via a bottom inlet 14 thereof, where it comes into contact with a volume of liquid 16 comprising a sulfide scavenger system (not separately labeled). The gas, having been treated by the liquid, flows out of a top end 18 of gas treatment vessel 6 and flows into sulfide measurement device 8 via treated gas outlet piping 20.

In one embodiment, the gas contained in gas source 2 consists substantially of about 15% H₂S, and about 3% CO₂, with the remainder being Methane. In one embodiment, gas flow from the flow regulator 4 is controlled such that the gas is flowed into the gas treatment vessel 6 at a flow rate of 100 mL/min. up to 500 mL/min. In one embodiment, a gas sparging device, such as a fine pore frit (not shown), is utilized to better distribute the gas into the scavenger liquid. In one embodiment, the gas treatment vessel 6 contains about 20 g of a liquid H₂S scavenger, such as one or more diaminols/diaminacetals in a chemical system as described above. In one aspect, to the extent possible, H₂S and/or CO₂ are scavenged from the gas by the scavenger and the treated (“sweet”) gas exits top end 18 of treatment vessel 6. In one embodiment, sulfide measurement device 8 monitors the concentration of H₂S leaving treatment vessel 6. in the gas phase. In other embodiments (not shown), a CO₂ measurement device may be similarly employed to monitor the concentration of CO₂ leaving treatment vessel 6 in the gas phase.

In one embodiment, gas is flowed through sulfide scavenger testing system 100 until the outlet H₂S concentration reaches a 4,000 ppm target, at which point the test is concluded. This relative time duration before the target is reached is used to compare H₂S scavenger capacity given the kinetics of each sulfide scavenger system. The longer the duration, the better the capacity.

### Long Reaction Time Capacity Test

In this test, methane containing 15% H₂S and 3% CO₂ by weight is sparged via a fine pore frit (not shown) at 100 mL/min through a column of 20 g of scavenger solution (See FIG. 1). The fine bubbles produced rise slowly, providing plenty of time, plenty of surface area, and a short path through which the H₂S must diffuse to the scavenger in the liquid 16. Detector 8 on the outlet line 20 is used to measure H₂S concentration in the exiting gas. The times to first detection (FD), at a threshold of 15 ppm, and to complete breakthrough (BT), at the detector saturation of 4000 ppm (0.4%), are recorded. This test is designed to measure the effective scavenging capacity in applications with long contact times.

Note that the true total capacity of a scavenger with infinite time to react is simply equal to the stoichiometry of the reaction—the moles of H₂S consumable per mole of scavenging molecule, which can be calculated. This Long Reaction Time Capacity measurement falls short of that ultimate stoichiometric capacity and so is still reaction rate dependent, just less so than the Short Reaction Time Capacity Test.

### Short Reaction Time Capacity Test

This test is the same as the Long Reaction Time Capacity Test, but without the gas sparging device, so as to produce larger bubbles, and at a higher feed rate of 200 mL/min, after the first 8 minutes at 100 mL/min. The larger bubbles rise quickly through the liquid 16, providing little time, little surface area, and a long path through which the H₂S must diffuse to the scavenger in the liquid. This test is designed to measure the effective scavenging capacity in applications with short contact times. It is far more reaction rate dependent than the Long Reaction Time Capacity and so is a more critical and differentiating test.

One thing to note when comparing the present invention to triazine is that scavenging reaction rates are a function of reactant concentrations, and the irreversible (scavenging) reaction of hydrogen sulfide involves two successive reactions: first to the thiol, then to the thioether. With triazine, one H₂S reacts twice with the same molecule of triazine, replacing one amine, so the rate is “first order”, meaning proportional to the concentration of triazine. In one aspect, for the diaminols and diaminacetals of the present invention, each H₂S must react with two different molecules of diaminol or diaminacetal, replacing two amines, so the rate is “second order”, meaning proportional to the square of the concentration of diaminol or diaminacetal. Thus, as the concentration of active triazine is depleted by say 50%, the scavenging rate is reduced by 50%, and depletion of 75% reduces it 75%. In contrast, if the concentration of active diaminol or diaminacetal is depleted by 50%, the rate is reduced by 75% (1-0.5²), and depletion of 75% reduces it 94% (1-0.25²). This more extended period of reduced rates extends the duration between the time of first detection (FD) and the time of breakthrough (BT) from about one minute for triazine to about ten minutes for the diaminol or diaminacetal. This reduced rate of reaction due to loss of active concentration should not be confused with a lower “rate constant” for the reaction as a function of concentration.

### Storage Capacity Loss Test

This test compares the scavenging capacity after 24 hours of storage of the scavenger system at room temperature to that after 72 hours of storage thereof at room temperature. In one embodiment, a sample of the scavenger system is made fresh and split in two portions. The Short Reaction Time Capacity Test is run on one portion after standing 24 hours on the bench at room temperature. Then the same test is run on the other portion after standing 72 hours on the bench at room temperature. The two measured capacities are compared to see if the product degrades in storage. Importantly, some scavengers lose activity with time due to secondary reactions and re-equilibrations that degrade their active form.

### Solution Homogeneity Test

In one aspect, as two-phase reaction products are problematic, scavenger system homogeneity is desired. The two phases could be emulsified or otherwise finely dispersed, but in practice, even fine, seemingly stable dispersions eventually separate or stratify in extended storage or critical use and are not robust enough for practical use. They are not allowed, for example, to be fed through capillaries for fear of plugging them. In this pass/fail test, a sample is held up for visual inspection in a backlit clear vial. A clear, single-phase, homogeneous solution is a pass. Anything else is a fail.

### Hot Spray Fouling Test

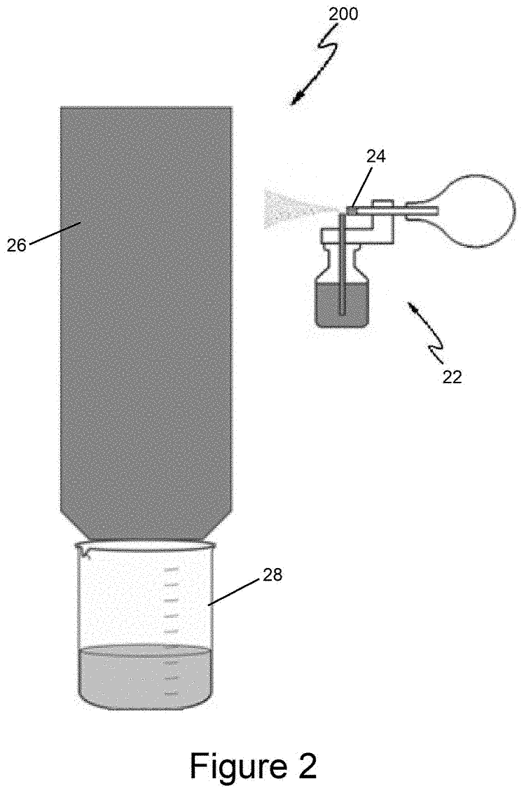

In many gas applications, the scavenger must by sprayed into a hot gas stream. This can create insoluble materials (“solids”) as the scavenger system evaporates and/or decomposes. These solids accumulate and foul (interfere with the function) of the vessel, line, or equipment to which the scavenger is being fed. This precludes the scavenger being used in that application. Referring now to FIG. 2, a spray test system 200 is utilized to for the Hot Spray Fouling Test. In one embodiment, an injection atomizer apparatus 22 is employed, wherein the scavenger formulation (not separately labeled) is sprayed onto a sheet 26 through an atomizing nozzle 24, like that used to introduce the scavenger into a gas line, tank, or vessel. The material is first placed in a double boiler (not shown) and brought to 180° F. The heated liquid is then placed in the injection atomizer apparatus 22 and sprayed horizontally onto an aluminum sheet 26 twelve inches away. The chemical drains gravitationally down the sheet where it is funneled into a beaker 28 for collection. If solids are observed on the sheet 26 or in the draining or collected fluid, the sample fails. If no solids are observed, it passes the test.

### Hot Spray Degradation Test

Scavenger systems that pass the Hot Spray Fouling Test are then tested quantitatively to measure any detectable degradation in activity that may have occurred during that test. In the Hot Spray Degradation Test, the collected, sprayed fluid from the Hot Spray Fouling Test is retested for scavenging capacity in the Long Reaction Time Capacity Test and compared to its pre-spray capacity in that test. In one aspect, at least 80% capacity must remain to pass this test. Passing this test ensures the scavenger will not be excessively lost or degraded by heat and evaporation at the application point.

### Spent Solids Fouling Test

In every application, the active component of the scavenger system reacts with H₂S to from a reaction product. Once all the active scavenger has been so reacted, it is spent. For example, the spent scavenger reaction product in aldehyde-amine based scavengers have most of the amines replaced in various ways by thioethers. Thioethers are much less water soluble than amines, so the reaction products are often insoluble. Thioether bridges also connect spent scavenger molecules to each other, thereby forming insoluble polymeric solids. These solids accumulate and foul (interfere with the function) of the vessel, line, or equipment to which the scavenger is being fed. To test for this, after s scavenger system has been subjected to the Long Reaction Time Capacity Test is done, the gas treatment vessel 6 (which may be constructed from a transparent material, such as glass) is held up to a light for visual inspection. If any solids or insoluble viscous liquids are observed poorly dispersed in the spent scavenger system or clinging to the sides of the vessel, the scavenging system employed in that Long Reaction Time Capacity Test fails this test. A homogeneous, single-phase solution or thin layer of readily flowable non-sticking liquid constitutes a pass.

### Spent Water Flowability Test

In field applications, spent scavenger is disposed of through a wastewater system. Spent scavenger might be soluble in the scavenger system but insoluble in wastewater. If it cannot be disposed of, it is not suitable for use. To ensure wastewater processing runs trouble free, spent scavenger passing the Spent Solids Fouling Test is then added to 39 times its weight in water (2.5%) and examined again for insoluble solids and viscous, sticky liquids that might foul the wastewater system. Any solids or thick, sticky insoluble liquid is a fail. A homogeneous, single-phase solution or thin layer of readily flowable non-sticking liquid constitutes a pass.

### Test Results

### Scavenging Ability of Diimine vs. Diaminacetal

Table 2 compares the performance of the straight glyoxal isopropyl diimine (GID) to various glyoxy isopropyl diaminacetals (GIDAc). In one embodiment, GID was prepared in the previously described manner by reacting 1 mole of glyoxal with 2.1 moles of isopropylamine and dehydrating the reaction product. The GID was then added to various reactive and unreactive solvents. Solvents included hydrocarbons, hexane and xylene, which are unreactive to imine and sulfide; methyl isobutyl ketone, which is unreactive to imine but reactive toward sulfide anion; and alcohols, methanol, isopropanol, butanol and heptanol, which are reactive to imine but unreactive toward sulfide. The components, their weight ratios, and reaction equivalent weights (Eq. Wt.) are listed at the top of Table 2. The number of molar equivalents in the 20 g test sample is then listed in terms of the imine (GID) and its aminacetal (GIDAc) assuming every mole of alcohol available reacts with the GID. Thus, the GID added to xylene remains all GID. The GID added to excess mole equivalents of methanol (MeOH) is all converted to GIDAc. Alcohols other than straight MeOH produce a mixture of GID and GIDAc. These compositions were run in the Short Reaction Time Capacity Test and the minutes to first detection (FD) and to breakthrough (BT) recorded. The minutes per equivalent (min/Eq.) for the FD and BT were then calculated. Finally, a predictive equation (Predicted FD or BT) for the capacity of each composition was derived based on its concentrations of GID, GIDAc, and MIBK (methyl isobutyl ketone) equivalents. The best fit equations were FD=92*(Eq. GID)+324*(Eq. GIDAc)+515*(Eq. MIBK) and BT=102*(Eq. GID)+404*(Eq. GIDAc)+601*(Eq. MIBK). The coefficient for each parameter in these equations indicates the size of that parameter's contribution to the overall result. Thus, while the GID contributed 92 minutes per equivalent to the FD capacity, the GIDAc contributed 324 minutes per equivalent to the FD capacity. Similarly, the GID contributed 102 minutes per equivalent to the BT capacity, and the GIDAc contributed 404 minutes per equivalent to the FD capacity. The only significant deviation from this general trend was with the heptanol, which, because it has the highest equivalent weight, was predicted to be the worst of the alcohols, and it was the worst of the alcohols, but even so, was worse than predicted. It is possible larger, higher alcohols are less efficient at forming acetals. The apparent boost from the MIBK may come from mediating and accelerating the transfer of bisulfide anion to the imine.

The bottom section of Table 2 lists the results of the other qualifying tests: Solution Homogeneity, Hot Spray Fouling, Hot Spray Degradation, Spent Solids Fouling, and Spent Water Flowability. An “X” indicates the test was not done because the prior test had failed. Hexane failed the Solution Homogeneity Test to the point its capacity could not even be tested. Xylene was hazy but could be tested. The only one to pass all the tests contained ⅔ GID and ⅓ heptyl GIDAc, but as noted, that was the worst in the capacity test, having only one fifth the capacity of the pure methyl GIDAc.

TABLE 2

| Chemical Name | Abrev. | Eq. Wt. | Grams in Test (20 g total) | | | | | | | | |
|---|---|---|---|---|---|---|---|---|---|---|---|
| Glyoxal Isopropyl Diimine | GID | 70.11 | 13 | 13 | 13 | 13 | 13 | 13 | 13 | 13 | 13 |
| Hexane | Hexane | | 7 | | | | | | | | |
| Xylene | Xylene | | | 7 | | | | | | | |
| Methyl Isobutyl Ketone | MIBK | 100.16 | | | 7 | | | | | | |
| Methanol | MeOH | 32.04 | | | | 7 | 4 | | 4 | | |
| Isopropanol | i-PrOH | 60.10 | | | | | | 7 | | | |
| n-Butanol | n-BuOH | 74.12 | | | | | 3 | | | 7 | |
| n-Heptanol | n-C₇OH | 116.20 | | | | | | | 3 | | 7 |
| | | | Molar Equivalents in Test | | | | | | | | |
| Glyoxal Isopropyl Diimine | GID | Eq./test | 0.185 | 0.185 | 0.185 | | 0.020 | 0.069 | 0.035 | 0.091 | 0.125 |
| Glyoxy Isopropyl Diaminacetal | GIDAc | Eq./test | | | | 0.185 | 0.165 | 0.116 | 0.151 | 0.094 | 0.060 |
| Methyl Isobutyl Ketone | MIBK | Eq./test | | | 0.070 | | | | | | |

| | | | | | | | | | | |
|---|---|---|---|---|---|---|---|---|---|---|
| TOTAL | Eq./test | 0.185 | 0.185 | 0.255 | 0.185 | 0.185 | 0.185 | 0.185 | 0.185 | 0.185 |

| Test Parameter | Test Results | | | | | | | | |
|---|---|---|---|---|---|---|---|---|---|
| Short Reaction Time Capacity First Detection (min) | X | 17 | 53 | 60 | 62 | 48 | 56 | 54 | 12 |
| Short Reaction Time Capacity Breakthrough (min) | X | 19 | 61 | 75 | 70 | 60 | 64 | 61 | 15 |
| min/Eq. FD | X | 92 | 208 | 324 | 334 | 259 | 302 | 291 | 65 |
| min/Eq. BT | X | 102 | 239 | 404 | 378 | 324 | 345 | 329 | 81 |
| Predicted FD (min) | X | 17 | 53 | 60 | 55 | 44 | 52 | 39 | 31 |
| Predicted BT (min) | X | 19 | 61 | 75 | 69 | 54 | 64 | 47 | 37 |
| Solution Homogeneity | FAIL | FAIL | PASS | PASS | PASS | PASS | PASS | PASS | PASS |
| Hot Spray Fouling | X | FAIL | FAIL | FAIL | PASS | FAIL | PASS | PASS | PASS |
| Hot Spray Degradation | X | X | X | X | PASS | X | PASS | PASS | PASS |
| Spent Solids Fouling | X | FAIL | PASS | PASS | PASS | PASS | PASS | PASS | PASS |
| Spent Water Flowability | X | X | FAIL | FAIL | FAIL | FAIL | FAIL | FAIL | PASS |

### Scavenging Ability of Diaminacetal vs. Diaminol

Tables 3 and 4 depict a comparison of the performance of the glyoxy isopropyl diaminacetal (GIDAc) to that of the glyoxy isopropyl diaminol (GIDA) in both the Long and the Short Reaction Time Capacity Tests, respectively. GIDAc was prepared by reacting 1 mole of glyoxal (as 40% aqueous sol.) with 2.1 moles of isopropylamine, dehydrating the reaction product, then reacting it with an excess molar amount of MeOH. The weight ratios as GID and MeOH, regardless of final form, for the 20 g test are noted in the Tables. GIDA was prepared by reacting 1 mole of glyoxal (as 40% aqueous sol.) with 2.1 moles of isopropylamine in a sufficient amount of MeOH and BuOH to keep the product from separating into an aqueous layer and a non-aqueous layer. The weight ratios as GID, Water, MeOH and BuOH, regardless of actual form, are noted in the Tables. For comparison, a 37.5% aqueous solution of MEA triazine (monoethanolamine-formaldehyde adduct), the conventional prior art scavenger, was also tested as a control. The reaction equivalent weight (Eq. Wt.) as GID or Triazine is noted and the molar equivalents per test (Eq/test) are calculated for each test sample. The capacity in terms of time in minutes to first detection (Min to FD) and to breakthrough (Min to BT) are recorded, and finally, the capacity in minutes per molar equivalent (Min/Eq) is calculated. This allows for comparison of the different chemistries on an equivalent molar basis

TABLE 3
Long Reaction Time Capacity Test

| Scavenger | Triazine | GIDAc | GIDAc | GIDA |
|---|---|---|---|---|
| g Triazine | 7.50 | | | |
| g as GID | | 13.00 | 9.00 | 6.68 |
| g as Water | 12.50 | | | 3.32 |
| g as MeOH | | 7.00 | 11.00 | 8.00 |
| g as BuOH | | | | 2.00 |
| Eq. Wt. | 109.6 | 70.1 | 70.1 | 70.1 |
| Eq/test | 0.068 | 0.185 | 0.128 | 0.076 |
| Min to FD | 66 | 105 | 76 | 46 |
| Min to BT | 68 | 131 | 99 | 61 |
| Min/Eq FD | 965 | 566 | 592 | 607 |
| Min/Eq BT | 994 | 707 | 771 | 805 |

TABLE 4
Short Reaction Time Capacity Test

| Scavenger | Triazine | GIDAc | GIDAc | GIDA | GIDA |
|---|---|---|---|---|---|
| g Triazine | 7.50 | | | | |
| g as GID | | 11.00 | 8.00 | 6.68 | 6.68 |
| g as Water | 12.50 | | | 3.32 | 3.32 |
| g as MeOH | | 6.00 | 9.00 | 7.50 | 8.00 |
| g as BuOH | | 3.00 | 3.00 | 2.50 | 2.00 |
| Eq. Wt. | 109.6 | 70.1 | 70.1 | 88.1 | 88.1 |
| Eq/test | 0.068 | 0.157 | 0.114 | 0.076 | 0.076 |
| Min to FD | 25 | 50 | 42 | 35 | 39 |
| Min to BT | 26 | 59 | 50 | 41 | 46 |
| Min/Eq FD | 365 | 319 | 368 | 462 | 515 |
| Min/Eq BT | 380 | 376 | 438 | 541 | 607 |

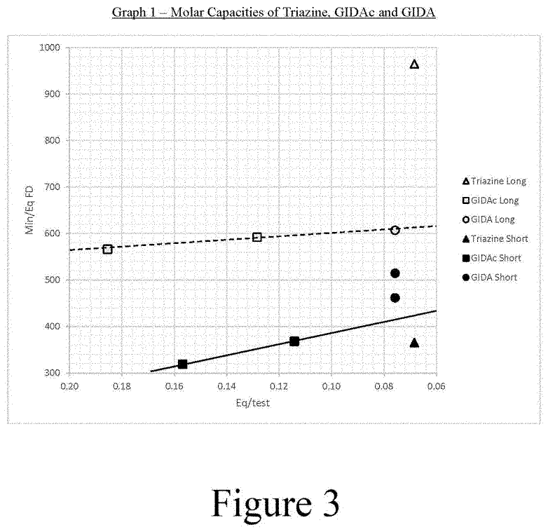

As noted above, the measured capacity in a dynamic scavenging reaction test is a function of reaction time and molar concentration. If you were to hold the total moles constant and add more inert solvent, the reaction time, and thus the measured capacity, would increase for same total number of moles. If you were to then remove some of that diluted solution to return to the original volume, the reaction time and thus the measured capacity would then decrease to about the original number, but the solution would now have fewer total moles—the measured capacity per mole thus increases when the molar concentration is reduced. This effect must be accounted for when comparing chemicals at different molar concentrations. In Graph 1 (depicted in FIG. 3) is plotted the molar capacity against molar concentration, using a reverse scale. As can be seen in Graph 1, lower concentrations have predictably higher capacities. Accounting for this, triazine has a greater molar capacity than GIDAc at long reaction times, but a lower molar capacity at short reaction times. This suggests triazine reacts slower than GIDAc under these comparable reaction conditions. Similarly, GIDAc has the same capacity as GIDA at long reaction times, but a lower molar capacity at short reaction times. This suggests GIDAc reacts slower than GIDA under these comparable reaction conditions. Not to be bound by theory it is believed this is because the hydroxyl substituent on the reactive carbon of the aminol is more electron withdrawing than the alkoxyl substituent, and this further activates the carbon and allows it to react faster with the H₂S.

### Amine Substituent Effects

Table 5 compares different alkyl substituents on glyoxy alkyl diaminols. These compounds were prepared by reacting one mole of glyoxal, as a 40% aqueous solution, with 2.1 moles of various primary amines in a sufficient amount of MeOH to keep the product from separating into an aqueous layer and a non-aqueous layer. For each amine substituent: methyl (Me), ethyl (Et), normal-propyl (n-Pr), iso-propyl (i-Pr), and iso-propanol [1-propan-2-ol] (i-PrOH), the presence or absence of alpha branching on that substituent is noted. The weight ratios of the diaminols, water, and MeOH, are listed, along with their equivalent weights (Eq. Wt.) and the total equivalents per 20 g test (Eq/test). To measure the product stability, the Storage Capacity Loss between 24 and 72 hours was determined. As described earlier, a fresh sample of the scavenger system is split in two portions. The Short Reaction Time Capacity Test is run on one portion after standing 24 hours on the bench at room temperature. Then the same test is run on the other portion after standing 72 hours on the bench at room temperature. Both the 24-hr and 72-hr capacities (Min to FD and BT) and molar capacities (Min/Eq FD and BT) are listed in the table with the percent capacity loss for each substituent calculated below it. Only the glyoxy isopropyl diaminol, the one containing an alpha-branched substituent, showed no degradation. The others lost 34% to 77% of their scavenging capacity in 48 hours (72-24), presumably due to secondary reactions and re-equilibrations that degraded their active form. In this system employing only methanol, all of these compositions failed both the Hot Spray Fouling Test and the Spent Water Flowability Test. Two of them, the iso-propylamine [propan-2-amine] and the iso-propanolamine [1-aminopropan-2-ol] passed the Spent Solids Fouling Test.

TABLE 5
Amine Substituent Effect on Degradation and Fouling

| Sample Age | Substituent | Me | Et | n-Pr | i-Pr | i-PrOH |
|---|---|---|---|---|---|---|
| | α-Branching | no | no | no | yes | no |
| | g Diaminol | 5.79 | 6.29 | 6.68 | 6.68 | 7.03 |
| | g Water | 4.21 | 3.71 | 3.32 | 3.32 | 2.97 |
| | g MeOH | 10.00 | 10.00 | 10.00 | 10.00 | 10.00 |
| | Eq. Wt. | 60.1 | 74.1 | 88.1 | 88.1 | 104.1 |
| | Eq/test | 0.096 | 0.085 | 0.076 | 0.076 | 0.068 |
| 24 Hrs | Min to FD | 15 | 31 | 31 | 31 | 13 |
| | Min to BT | 29 | 37 | 38 | 40 | 30 |
| 72 Hrs | Min to FD | 8 | 9 | 7 | 31 | 6 |
| | Min to BT | 19 | 20 | 17 | 41 | 10 |
| 24 Hrs | Min/Eq FD | 156 | 365 | 409 | 409 | 192 |
| | Min/Eq BT | 301 | 436 | 501 | 528 | 444 |
| 72 Hrs | Min/Eq FD | 83 | 106 | 92 | 409 | 89 |
| | Min/Eq BT | 197 | 236 | 224 | 541 | 148 |
| 72- | FD Capacity Loss | 47% | 71% | 77% | 0% | 54% |
| 24 Hrs | BT Capacity Loss | 34% | 46% | 55% | −2% | 67% |

| | | | | | |
|---|---|---|---|---|---|
| Solution Homogeneity | Pass | Pass | Pass | Pass | Pass |
| Hot Spray Fouling | Fail | Fail | Fail | Fail | Fail |
| Spent Solids Fouling | Fail | Fail | Fail | Pass | Pass |
| Spent Water Flowability | Fail | Fail | Fail | Fail | Fail |

Not to be bound by theory, it is believed that the degradation in activity occurs via the dimerization and oligomerization of the cis conformation of the diaminol, and that this conformation is inhibited by the steric crowding of the alpha branching.

### Solvent System Effects

In one aspect, it is desirable that a solvent system serves three competing purposes:
1. Preventing the aminol from separating into an imine and water.
2. Solvating and preserving the activity of the aminol under hot, evaporative spray conditions.
3. Dissolving the expended scavenger reaction products and rendering them flowable in water.

Table 6 below summarizes the results of tests to find a system to satisfy all requirements for the reaction of 1 mole glyoxal and 2 moles isopropylamine. The two-phase reaction product comprising GID and water is combined with the various alcohols and the overall composition, in percent, is listed in the table. The alcohols are divided into two groups: water miscible alcohols, those with a C/O ratio of 3 or less and an octanol-water partition logarithm, LogP, of 0.8 or less; and water immiscible alcohols, those with a C/O ratio of 4 or greater and an octanol-water partition logarithm, LogP, of 0.9 or greater. The alcohols can also be divided into high volatility alcohols, those with higher (“Hi”) vapor pressure (VP), like methanol and ethanol; and low volatility alcohols, those with lower vapor pressure (VP), like the glycols (e.g. propylene glycol, hexylene glycol), glycol ethers (e.g. 2-butoxy ethanol, aka ethylene glycol mono-butyl ether or EGMBE) and the higher (carbon number) alcohols (e.g. butanol, heptanol, etc.). The C/O ratio, VP category, and LogP are listed for each alcohol as well as for GID and water. For each alcohol blend, the total percentage of low volatility alcohol in the overall formulation and the ratio of water immiscible alcohols to prehydrated GID is listed. The formula percent weighted ratio of carbon to oxygen (C/O) in the solvent, as defined earlier, is also listed. Again, this is simply the sum of each alcohols percent in the total scavenger system multiplied by its C/O ratio, so, for 50% MeOH (C/O=1), it is 50%×1=0.50. The table then lists which tests each a scavenger system passed or failed. An X indicates a test was not done because the prequalifying test failed.

Several solvent system characteristics are apparent from Table 6. In the embodiments tested, a minimum amount of low volatility alcohol had to be incorporated to pass the hot spray tests. This was between 5.3% (pass) and 6.5% (fail). In addition, a minimum amount of water immiscible alcohol relative to the active component calculated as GID had to be added to pass the spent scavenger tests. This was at least 18% of the scavenger active component calculated as GID provided the formula percent weighted carbon to oxygen (C/O) was also at least 1.0. In one aspect, not all water miscible alcohols can keep GID and water together even if no water immiscible alcohol is added to induce phase separation thereof. Ethylene glycol and diethylene glycol (DEG) do not work. Likely, similar solvents, like triethylene glycol (TEG) and sorbitol are in the same group. These partition to octanol from water less than water itself (LogP<−1.4). Of those water miscible alcohols between LogP-1.4 and 0.8, which do work, it takes from 20% to 60% of the total scavenger system to keep the GID, the water, and the higher alcohol in a single phase. Given the minimum amount of water immiscible alcohol needed, this translates to a formula percent weighted ratio of carbon to oxygen (C/O) in the solvent of between 1.0 and 2.0.

TABLE 6
Mixed Alcohol Glyoxy Isopropyl Diaminol Formulations

| Material | | C/O | VP | LogP | % Composition | | | | | | | | | |
|---|---|---|---|---|---|---|---|---|---|---|---|---|---|---|
| Glyox. iPr. | GID | 4 | Lo | 2.3 | any | any | 37.9 | 26.5 | 37.8 | 27.9 | 34.2 | 34.2 | 32.1 | 35.5 |
| Diimine | | | | | | | | | | | | | | |
| Water | H2O | 0 | Lo | −1.4 | any | any | 33.6 | 23.5 | 33.6 | 24.7 | 30.3 | 30.3 | 28.5 | 31.4 |
| Ethylene Glycol | EG | 1.0 | Lo | −1.7 | Fail | | | | | | | | | |
| Diethylene | DEG | 1.3 | Lo | −1.5 | | Fail | | | | | | | | |
| Glycol | | | | | | | | | | | | | | |
| Propylene | PG | 1.5 | Lo | −1.3 | | | | | | | | | | |
| Glycol | | | | | | | | | | | | | | |
| Methanol | MeOH | 1.0 | Hi | −0.7 | | | 28.5 | 50.0 | 21.4 | 42.1 | 25.8 | 29.0 | 30.3 | 23.1 |
| Ethanol | EtOH | 2.0 | Hi | −0.2 | | | | | | | | | | |
| Hexylene | HG | 3.0 | Lo | 0.0 | | | | | | | | | | |
| Glycol | | | | | | | | | | | | | | |
| 2-Butoxy | EGMBE | 3.0 | Lo | 0.8 | | | | | | | | | | |
| Ethanol | | | | | | | | | | | | | | |
| n-Butanol | nBuOH | 4.0 | Lo | 0.9 | | | | | 7.2 | | | | | |
| n-Heptanol | n-C₇OH | 7.0 | Lo | 2.5 | | | | | | | 9.7 | | | |
| 2-Ethylhexanol | 2EHOH | 8.0 | Lo | 2.8 | | | | | | 5.3 | | | | 10.0 |
| n-Octanol | n-C₈OH | 8.0 | Lo | 3.0 | | | | | | | | | 9.1 | |
| Neodol ® 91 | n-C₁₀OH | 10.0 | Lo | 4.1 | | | | | | | | 6.5 | | |
| % Low Volatility Alcohol | | | | | | | 0.0 | 0.0 | 7.2 | 5.3 | 9.7 | 6.5 | 9.1 | 10.0 |
| Higher-Alcohol/GID (%) | | | | | X | X | 0.0 | 0.0 | 19.0 | 18.8 | 28.4 | 19.0 | 28.3 | 28.3 |
| Formula % Weighted C/O | | | | | X | X | 0.3 | 0.5 | 0.5 | 0.8 | 0.9 | 0.9 | 1.0 | 1.0 |
| Solution Homogeneity | | | | | Fail | Fail | Pass | Pass | Pass | Pass | Pass | Pass | Pass | Pass |
| Hot Spray Fouling | | | | | X | X | Fail | Fail | Pass | Fail | Pass | Pass | Pass | Pass |
| Hot Spray Degradation | | | | | X | X | X | X | Pass | X | Pass | Pass | Pass | Pass |
| Spent Solids Fouling | | | | | X | X | Fail | Fail | Pass | Pass | Pass | Fail | Pass | Pass |
| Spent Water Flowability | | | | | X | X | Fail | Fail | Fail | Pass | Pass | X | Pass | Pass |
| Material | | C/O | VP | LogP | % Composition | | | | | | | | | |
| Glyox. iPr. | GID | 4 | Lo | 2.3 | 25.3 | 24.5 | 24.1 | 25.3 | 29.5 | 32.1 | 26.4 | 28.6 | 26.1 | 21.3 |
| Diimine | | | | | | | | | | | | | | |
| Water | H2O | 0 | Lo | −1.4 | 22.3 | 21.8 | 21.3 | 22.3 | 26.1 | 28.5 | 23.4 | 25.4 | 23.1 | 18.9 |
| Ethylene Glycol | EG | 1.0 | Lo | −1.7 | | | | | | | | | | |
| Diethylene | DEG | 1.3 | Lo | −1.5 | | | | | | | | | | |
| Glycol | | | | | | | | | | | | | | |
| Propylene | PG | 1.5 | Lo | −1.3 | | 49.1 | 50.0 | | | | | | | |
| Glycol | | | | | | | | | | | | | | |
| Methanol | MeOH | 1.0 | Hi | −0.7 | 38.1 | | | 38.1 | 36.1 | 30.3 | | | | |
| Ethanol | EtOH | 2.0 | Hi | −0.2 | | | | | | | 45.2 | | | |
| Hexylene | HG | 3.0 | Lo | 0.0 | | | | | | | | 40.6 | 45.9 | |
| Glycol | | | | | | | | | | | | | | |
| 2-Butoxy | EGMBE | 3.0 | Lo | 0.8 | 9.5 | | | | | | | | | 55.8 |
| Ethanol | | | | | | | | | | | | | | |
| n-Butanol | nBuOH | 4.0 | Lo | 0.9 | | | | 9.5 | | | | | | |
| n-Heptanol | n-C₇OH | 7.0 | Lo | 2.5 | | | | | | | | | | |
| 2-Ethylhexanol | 2EHOH | 8.0 | Lo | 2.8 | 4.8 | 4.6 | 4.5 | 4.8 | | | 5.0 | 5.4 | 4.9 | 4.0 |
| n-Octanol | n-C₈OH | 8.0 | Lo | 3.0 | | | | | | | | | | |
| Neodol ® 91 | n-C₁₀OH | 10.0 | Lo | 4.1 | | | | | 8.3 | 9.1 | | | | |

| | | | | | | | | | | |
|---|---|---|---|---|---|---|---|---|---|---|
| % Low Volatility Alcohol | 14.3 | 53.7 | 54.6 | 14.3 | 8.3 | 9.1 | 5.0 | 46.0 | 50.8 | 59.8 |
| Higher-Alcohol/GID (%) | 18.8 | 18.9 | 18.8 | 56.5 | 28.1 | 28.3 | 18.9 | 18.9 | 18.8 | 18.9 |
| Formula % Weighted C/O | 1.0 | 1.1 | 1.1 | 1.1 | 1.2 | 1.2 | 1.3 | 1.7 | 1.8 | 2.0 |
| Solution Homogeneity | Pass | Pass | Pass | Pass | Pass | Pass | Pass | Pass | Pass | Pass |
| Hot Spray Fouling | Pass | Pass | Pass | Pass | Pass | Pass | Fail | Pass | Pass | Pass |
| Hot Spray Degradation | Pass | Pass | Pass | Pass | Pass | Pass | X | Pass | Pass | Pass |
| Spent Solids Fouling | Pass | Pass | Pass | Pass | Pass | Pass | Pass | Pass | Pass | Pass |
| Spent Water Flowability | Pass | Pass | Pass | Pass | Pass | Pass | Pass | Pass | Pass | Pass |

### Operation

In certain embodiments, the aminol/aminacetal or diaminol/aminacetal systems may be used to remove sulfides in upstream applications. In various applications, sulfides may be scavenged by the diaminol/aminacetal systems from the oil or natural gas through injection or in-situ formation into such equipment as contact/scrubber tower, direct line injection, batch treating, capillary or umbilical injection. In other applications, embodiments of H₂S scavengers of the present invention may be employed where fluid separation takes place, such as in intermediate storage lines and vessels, separators and fractioning equipment, transport lines and storage tanks. In other cases, sulfide impurities are removed from the hydrocarbon fluids and/or water streams during the refining process.

In one embodiment, H₂S scavenging with aminol/aminacetals or diaminol/diaminacetals, such as but not limited to, glyoxy isopropyl diaminol/diaminacetal, is performed upstream on mixed production at the wellhead, where oil, water, and/or natural gas is present and H₂S needs to be removed. In one embodiment, this may include a contact or bubble tower possible downstream of separated production.

In other embodiments, the aminol/aminacetal or diaminol/diaminacetal systems may be used to remove sulfides in midstream applications. In midstream applications, sulfides may be scavenged from the oil, natural gas or water through injection or in-situ formation into such equipment as contact/scrubber tower, direct line injection, batch treating, capillary or umbilical injection. Examples of this application are a direct line injection where the scavenger is injected directly into the oil, water, and/or gas transporting line downstream of the initial production location, as would be understood by one skilled in the art. In other embodiments, scavenging is performed in bubble tower/contact tower applications downstream of initial production locations.

In other embodiments, the aminol/aminacetal and diaminol/diaminacetal systems may be used to remove sulfides in downstream applications. In downstream applications, sulfides may be scavenged by the scavenger systems from the oil, natural gas or water through injection or in-situ formation into such equipment as contact/scrubber tower, direct line injection, batch treating, capillary or umbilical injection. In one embodiment, this may take place in a refinery by introduction of a scavenger into a fluid hydrocarbon stream where H₂S is present and needs to be removed and/or in a refinery where associated fluids are routed through a bubble/contact tower to remove H₂S.

In operational testing in a natural gas production facility, both an anhydrous glyoxy isopropyl aminacetal (GIDAc) scavenging system and an aqueous glyoxy isopropyl diaminol (GIDA) scavenging system were tested in comparison with an industry standard triazine-based scavenger. The test results demonstrated that both the GIDAc and GIDA based systems are employable in scavenging H₂S from natural gas. Furthermore, the GIDA-based scavenging systems produced a significantly higher H₂S removal on a pound-per-pound basis than the triazine-based scavenging system.

### Method

An exemplary method of scavenging sulfides from a hydrocarbon and/or aqueous fluid comprises:

A Diaminol and/or Diaminacetal Provision Step, comprising providing a diaminol and/or diaminacetal, such as glyoxy isopropyl diaminol and/or its aminacetals with both lower and higher alcohols.

A Diaminol/Diaminacetal System Preparation Step, comprising providing the diaminol/aminacetal in combination with at least one of surfactants, dispersants, hydrotropes, additional alcohols and amines, demulsifiers, corrosion and scale inhibitors, and/or other sulfide scavengers and

A Diaminol/Diaminacetal System Provision Step, comprising providing the diaminol/aminacetal system in contact with a hydrocarbon fluid or water.

The foregoing method is merely exemplary, and additional embodiments thereof consistent with the teachings herein may be employed. In addition, in other embodiments, one or more of these steps may be performed concurrently, combined, repeated, re-ordered, or deleted, and/or additional steps may be added.

The foregoing description of the invention illustrates exemplary embodiments thereof. Various changes may be made in the details of the illustrated construction and process within the scope of the appended claims by one skilled in the art without departing from the teachings of the invention. Disclosure of existing patents, publications, and/or known art incorporated herein by reference is to the extent required to provide details and understanding of the disclosure herein set forth. The present invention should only be limited by the claims and their equivalents.

We claim:

1. A composition for scavenging sulfides comprising;
an aminol;
wherein:
said composition is employable for scavenging sulfides from a hydrocarbon fluid and/or an aqueous liquid.

2. The composition for scavenging sulfides of claim 1, wherein said composition is water soluble.

3. The composition for scavenging sulfides of claim 1, wherein said aminol comprises a diaminol.

4. The composition for scavenging sulfides of claim 3, comprising a glyoxy isopropyl diaminol.

5. The composition for scavenging sulfides of claim 1, comprising water.

6. The composition for scavenging sulfides of claim 1, comprising a solvent.

7. The composition for scavenging sulfides of claim 6, wherein said solvent comprises one or more alcohols.

8. The composition for scavenging sulfides of claim 7, wherein said solvent comprises at least one water miscible alcohol and at least one water immiscible alcohol.

9. The composition for scavenging sulfides of claim 7, comprising methanol.

10. The composition for scavenging sulfides of claim 1, wherein said composition, both before and after reacting with said hydrocarbon fluid sulfides, does not form solids.

11. The composition for scavenging sulfides of claim 10, wherein said composition, after reacting with sulfides in a hydrocarbon fluid, does not form solids when said spent composition is mixed with an aqueous liquid.

12. The composition for scavenging sulfides of claim 1, wherein said composition, both before and after reacting with said sulfides in an aqueous liquid, does not form solids in said aqueous liquid.

13. A composition for scavenging sulfides comprising an aminacetal.

14. The composition for scavenging sulfides of claim 13, wherein said composition is water soluble.

15. The composition for scavenging sulfides of claim 13, wherein said aminacetal comprises a diaminacetal.

16. The composition for scavenging sulfides of claim 15, comprising a glyoxy isopropyl diaminacetal.

17. The composition for scavenging sulfides of claim 16, comprising, methyl glyoxy isopropyl diaminacetal.

18. The composition for scavenging sulfides of claim 13, comprising water.

19. The composition for scavenging sulfides of claim 13, comprising a solvent.

20. The composition for scavenging sulfides of claim 19, wherein said solvent comprises one or more alcohols.

21. The composition for scavenging sulfides of claim 20, wherein said solvent comprises at least one water miscible alcohol and at least one water immiscible alcohol.

22. The composition for scavenging sulfides of claim 20, comprising methanol.

23. The composition for scavenging sulfides of claim 13, wherein said composition, both before and after reacting with hydrocarbon fluid sulfides, does not form solids.

24. The composition for scavenging sulfides of claim 23, wherein said composition, after reacting with said hydrocarbon fluid sulfides, does not form solids when said spent composition is mixed with an aqueous liquid.

25. The composition for scavenging sulfides of claim 13, wherein said composition, both before and after reacting with said sulfides in an aqueous liquid, does not form solids in said aqueous liquid.

26. A composition for scavenging sulfides, said composition produced by the reaction of one or more aldehydes in an aqueous solution with one or more primary amines and/or secondary amines, wherein the molar quantity of amine functional groups is equal to at least the molar quantity of aldehyde functional groups, wherein the water present in the aqueous reaction medium and water produced by said reaction between said one or more aldehydes and said one or more primary amines and/or secondary amines is maintained in the composition.

27. The composition for scavenging sulfides of claim 26, wherein said reaction of said one or more aldehydes and said one or more primary amines and/or secondary amines is carried out in the presence of one or more alcohols.

28. The composition for scavenging sulfides of claim 27, wherein said one or more alcohols comprises at least one water miscible alcohol and at least one water immiscible alcohol.

29. The composition for scavenging sulfides of claim 27, wherein one said alcohol is methanol.

30. The composition for scavenging sulfides of claim 26, wherein at least one said aldehyde comprises a dialdehyde.

31. The composition for scavenging sulfides of claim 30, wherein one said dialdehyde is glyoxal.

32. The composition for scavenging sulfides of claim 26, wherein each primary amine contains an alkyl group having alpha branching.

33. The composition for scavenging sulfides of claim 26, wherein one said primary amine is isopropyl amine.

34. The composition for scavenging sulfides of claim 26, wherein said composition, both before and after reacting with hydrocarbon fluid sulfides, does not form solids.

35. The composition for scavenging sulfides of claim 34, wherein said composition, after reacting with said hydrocarbon fluid sulfides, does not form solids when said spent composition is mixed with an aqueous liquid.

36. The composition for scavenging sulfides of claim 26, wherein said composition, both before and after reacting with said sulfides in an aqueous liquid, does not form solids in said aqueous liquid.

37. An improved composition for scavenging sulfides from a hydrocarbon fluid medium or an aqueous liquid medium, comprising:
at least one compound selected from the group consisting of:
an aminol;
a diaminol;
an aminacetal; and
a diaminacetal;
the improvement comprising a sulfide scavenging composition, that, both before and after reacting with said sulfides, does not form solids.

38. The improved composition for scavenging sulfides of claim 37, comprising a solvent.

39. The improved composition for scavenging sulfides of claim 37, comprising water.

40. The improved composition for scavenging sulfides of claim 38, wherein said solvent comprises one or more alcohols.

41. The improved composition for scavenging sulfides of claim 40, wherein said one or more alcohols comprises at least one water miscible alcohol and at least one water immiscible alcohol.

42. The improved composition for scavenging sulfides of claim 40, comprising methanol.

43. The improved composition for scavenging sulfides of claim 37, wherein said composition, after reacting with hydrocarbon fluid sulfides, does not form solids when said spent composition is mixed with an aqueous liquid.

44. The improved composition for scavenging sulfides of claim 37, comprising a glyoxy isopropyl diaminol and/or a glyoxy isopropyl diaminacetal.

45. A method of scavenging sulfides from a contained hydrocarbon fluid medium or a contained aqueous medium, comprising:
providing a scavenging composition comprising at least one compound selected from the group consisting of:
an aminol;
a diaminol;
an aminacetal; and
a diaminacetal; and
introducing said scavenging composition to said contained hydrocarbon fluid medium or said contained aqueous medium, whereby at least one said compound reacts with said sulfides, and wherein said composition, both before and after said reaction with said sulfides, does not form solids.

46. The method of scavenging sulfides of claim 45, wherein said scavenging composition, after reacting with said sulfides in said hydrocarbon fluid medium, does not form solids when said hydrocarbon fluid medium is mixed with an aqueous liquid.

47. The method of scavenging sulfides of claim 45, wherein said scavenging composition comprises a solvent.

48. The method of scavenging sulfides of claim 47, wherein said solvent comprises one or more alcohols.

49. The method of scavenging sulfides of claim 48, wherein said one or more alcohols comprises at least one water miscible alcohol and at least one water immiscible alcohol.

50. The method of scavenging sulfides of claim 48, comprising methanol.

51. The method of scavenging sulfides of claim 45, wherein said scavenging composition comprises water.

52. The method of scavenging sulfides of claim 45, wherein said scavenging composition comprises a glyoxy isopropyl diaminol and/or a glyoxy isopropyl diaminacetal.
